(19) United States
(12) Patent Application Publication
(10) Pub. No.: US 2009/0270949 A1
(43) Pub. Date: Oct. 29, 2009

(54) EXTERNAL STIMULUS FOR ESTABLISHING COMMUNICATION WITH IMPLANTABLE MEDICAL DEVICE

(75) Inventors: Scott L. Kalpin, Harris, MN (US); William J. Mitchell, Eden Prairie, MN (US); Patrick S. Leong, Chatsworth, CA (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/108,795

(22) Filed: Apr. 24, 2008

(51) Int. Cl. A61N 1/00

(52) U.S. Cl. 607/60

(57) ABSTRACT

The present invention relates to systems, devices and methods for accessing an implantable medical device when the security key is not known. The systems, methods and devices help to access an implantable medical device through a back door to permit non-secure communications. The systems, devices, and methods require short range external stimulus to indicate to the implantable medical device that a valid communication is about to be received.

### FIELD OF THE INVENTION

This disclosure relates to implantable medical devices. More particularly to communication between implantable medical devices and external devices, such as programmers.

### BACKGROUND OF THE INVENTION

Medical devices implanted in patients may communicate with devices external to the patients via distance telemetry, such as radio frequency telemetry. Such distance telemetry communications do not require the external device, such as a programmer, to be located in close proximity to the patient, and thus are more convenient than proximity telemetry communications. While distance telemetry provides convenience to the communication process, security and unintentional access is a concern. In some devices a security key may be utilized to limit communications. However, certain situations may require access to be granted to the implantable medical device when the security key is not known, such as during an emergency. In such cases a back door access to the implantable device may be desired.

### BRIEF SUMMARY OF THE INVENTION

The present disclosure presents methods, systems, and devices that provide transmission between an external device and an implantable medical device.

One embodiment may include a method associated with establishing communication between an implantable medical device and an external device, the method being performed by the implantable medical device and including receiving a short range external stimulus, recognizing the external stimulus as a valid indication of impending authorized communication with an external device, preparing to receive the authorized communication from the external device; and conducting communication with the external device.

Another embodiment may include an implantable medical device with a tap sensor and a processor operably coupled to the tap sensor wherein the processor is configured to receive an external stimulus from the tap sensor and to recognize the external stimulus as a valid indicator of impending authorized communication.

In another embodiment, an implantable medical device includes a stimulus sensor and a processor operably coupled to the stimulus sensor wherein the processor is configured to receive an external stimulus from the stimulus sensor and to recognize the external stimulus as a valid indicator of impending authorized communication.

### BRIEF DESCRIPTION OF THE DRAWINGS

FIGS. 1-2 are schematic block diagrams of representative systems.

FIGS. 3-4 are flow diagrams of representative methods.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION OF THE INVENTION

The present disclosure describes methods, systems and devices that provide back door access for external devices to access implantable medical devices. The back door access may be used in emergency or non-emergency situations such as when the security key is not available.

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

The teachings of the present disclosure may be applied to any implantable infusion device capable of telemetric communication through both distance and proximity telemetry. For example, the infusion device may be an implantable signal generator, such as a cardiac defibrillator, a cardiac pacemaker, a neurostimulator, a gastric stimulator, an implantable monitoring device, an implantable infusion device, or another similar device.

The teachings of the present disclosure may also be applied to any external device capable of telemetrically communicating with an implantable medical device. For example, external device may be a physician or patient programmer device, a monitoring device, or the like.

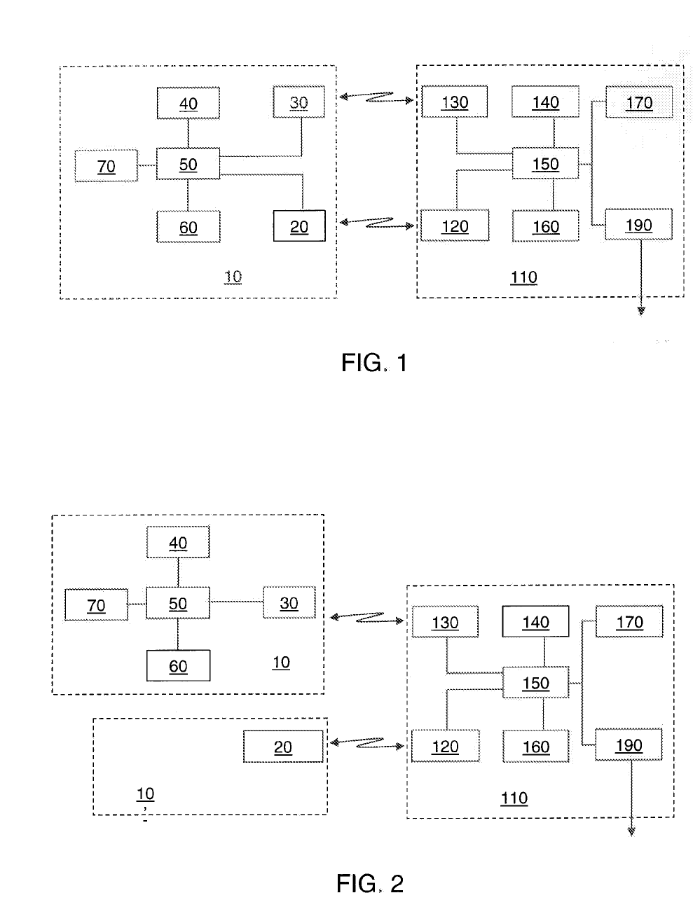

Referring now to FIGS. 1 and 2, schematic block diagrams of the two embodiments present invention system is shown. The system includes an implantable medical device 110 that is configured to be implanted in a patient and that has a hermetically sealed housing containing various operative components. In addition to a distance telemetry module 130 and an external stimulus sensor 120, a processor 150, power source 140, memory 160, clock 170, and therapy output module 190 are typically provided. However, device components provided to operate the implantable medical device 110 will vary depending upon the type of device and may include, for example, a pulse generator, capacitors, leads, sensors, pumping mechanisms, reservoirs, and various other components. The various electrical connections between components may also be changed depending on the desire system setup.

Processor 150 may be synchronous and typically operates on low power, such as a Motorola 68HC11 synthesized core operating with a compatible instruction set. Clock 170 may date/time stamp events and may be used for therapy control. Memory 160 includes memory sufficient for operation of device 110, such as volatile Random Access Memory (RAM) such as static RAM, nonvolatile Read Only Memory (ROM), Electrically Erasable Programmable Read Only Memory (EEPROM) such as Flash EEPROM, and register arrays configured on Application Specific Integrated Circuits (ASICs). Direct Memory Access (DMA) may be available to selected modules such as telemetry module 130 so that the selected modules can request control of a data bus and write data directly to memory 160 bypassing processor 150. External stimulus sensor 120 can be an accelerometer, a microphone, or other similar device that can accept a short range external stimulus as further discussed below.

Therapy output module 190 refers to components for carrying out the delivery or generation of therapeutic output to be delivered to a patient from implantable device 110. One of skill in the art will appreciate that the components may vary on a device-by-device basis and a therapy-by-therapy basis. For example, therapy module 190 may contain an oscillator if implantable medical device 110 is an electrical signal generator and may contain a pumping mechanism if device 110 is an infusion device.

Other components of implantable medical device 110 may include a system reset module, diagnostics module, sensor module or recharge module (not shown). In various embodiments, all components except the power source 140, which may be a battery, can be configured on one or more ASICs or may be one or more discrete components, or a combination of both. In various embodiments, all components, except the clock and power source, may be connected to a bi-directional data bus that is non-multiplexed with separate address and data lines.

Distance telemetry module 130 may include a transmitter, receiver, antenna, processor or other components necessary or desirable for carrying out distance telemetric communication. Distance telemetry typically refers to communications via radio frequency (RF) signals and includes telemetry M and telemetry C platforms. In general, distance telemetry communication may take place at distances of one meter or more, more typically over the range of about 3-20 meters. Of course, components of distance telemetry systems may communicate at distances of less than one meter. Distance telemetry modules are generally known in the art and various aspects are described in, for example, U.S. Pat. No. 6,240,317 issued to Villaseca et al. (May 29, 2001), and U.S. Pat. No. 6,482,154 issued to Haubrich et al. (Nov. 19, 2002).

It will be appreciated that a transceiver may be a discrete component that performs the functions of both the receiver and transmitter, and that the use of the latter terms will include the former.

The system illustrate din FIG. 1 may include an external device 10, which may be configured in a variety of ways. In the embodiment depicted in FIG. 1, external device 10 includes both a distance telemetry module 30 configured to communicate with implantable device 110 via telemetry module 130 and a stimulus module 20 configured to send a stimulus receivable by the external stimulus sensor 120 of the implantable device 1 10.

Stimulus module 20 can produce a stimulus receivable by external stimulus sensor 120 that indicates to the implantable device 110 that it is about to receive valid communications, as further discussed below. External device 10 may also include a processor 50, power source 40, memory 60, clock 70, or any other component necessary or desirable for operation of external device, including a display, a data input module, or the like. In other embodiments, external device 10 may not include stimulus module 20 if the stimulus is designed to be done by the patient or a person such as a doctor or nurse. For example, in one embodiment, no stimulus module 10 is required when the stimulus is tapping the implantable device 110.

In the embodiment depicted in FIG. 2, the system includes an external device 10′ containing stimulus module 20. External device 10′ is separate from external device 10, which includes distance telemetry module 30. In some embodiments, external device 10′ is a component or functionally attached to the external device 10, but in the present embodiment, devices 10, 10′ are contained in different housings. For example, external device 10′ may be a programming head and stimulus module 20 may be operably coupled to processor 50 wirelessly or via wired mechanism. While not shown, it will be understood that external device 10′ may include other components necessary or desirable for operation of external device 10′, such as a battery, regardless of whether external device 10′ is operably coupled to external device 10.

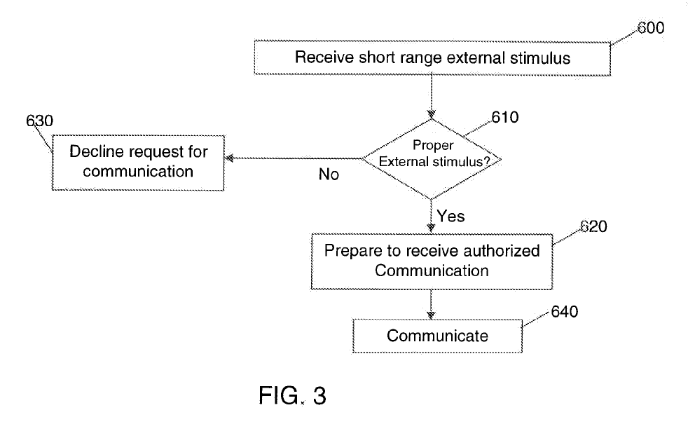

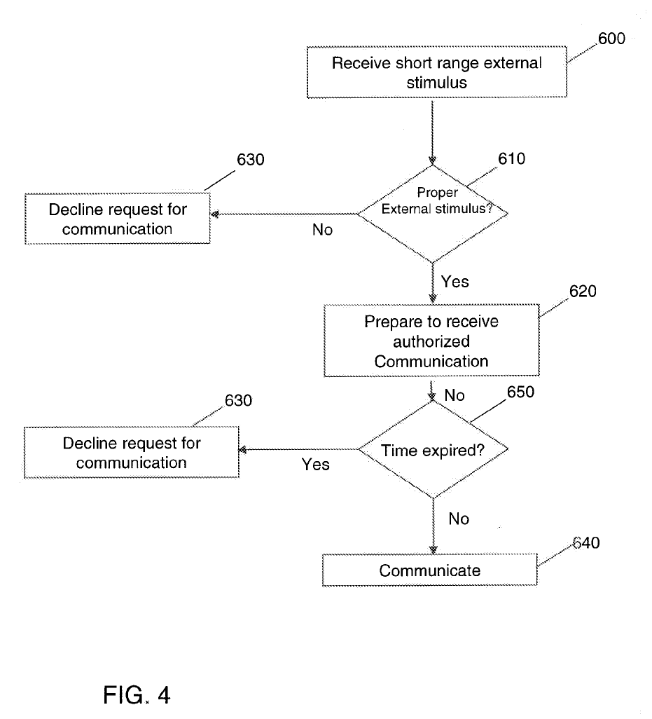

It will be understood that the components, devices and systems described with regard to FIGS. 1-2 are but examples of components, devices and systems that may be employed to engage in communication with the implantable medical device 110. However, for the sake of convenience, the discussion that follows with regard to FIGS. 3-4 will refer to devices and components as described with regard to FIGS. 1-2.

As previously stated, the present invention is directed towards situations where it may be necessary to access the implanted device when the security key is not known. As illustrated in FIG. 3, the present invention provides for communication with the implantable device 110 after a proximal security authorization is received by the implantable device 110. The proximal security authorization is a short range external stimulus. In the first step of the method the external stimulus may be received or sensed by the implantable device 110. The external stimulus indicates to the device that it is about to receive a valid request for non-secure communication (600) with an external device 10. The external stimulus may be, for example, tapping the patient in such a manner that is sensed by the implantable device 110. The tapping can be a simple string of taps or can be a tapped code. The patient or clinician may simply tap on the patient's skin located adjacent to the implanted medical device 110 in a set pattern. Control circuitry may be present that recognizes the proper tapping sequence and initiates the communication (as further described below). In other embodiments the external stimulus may be a signal produced by stimulus module 20 and received by stimulus sensor 120. In these further embodiments the external stimulus may be, for example, modulated or unmodulated magnetic fields. As may be appreciated, magnetic field parameters that can be varied include the clock rate of the field and the duty cycle of the field. The coded field may comprise a distinctive low speed variation, such as an on-off key at a predetermined rate. If the duty cycle is varied, it may be desirable for it to be varied by other than 50% to make it more difficult to unintentionally duplicate the coded signature. In still further embodiments audible or ultrasonic signals, either produced by the implanted device 110 or received by the implanted device 110, may be utilized.

After the external stimulus is received, the implantable device 110 must determine whether the external stimulus is a proper indication of impending authorized communication (610). Once the external stimulus is determined by the implantable device 110 to be a valid external stimulus, the implantable device 110 prepares to receive authorized communications (620). At this time the external device 10, capable of communication via distance telemetry, is used to communicate with the implantable device 110 (640). If the external stimulus is not verified then the implantable device 110 will decline the request to communicate (630). In the present embodiment, declining the request to communicate is, essentially, not preparing to receive communications.

In some embodiments the implantable device 110 may be limited in the information and programming functions that can be downloaded or uploaded using the non-secure link. In other embodiments the full range of programming may be available once the external stimulus is received. In still further embodiments the non-secure communication between the implantable device 110 and the external device 10 can be established and then a code or other encryption key can be exchanged such that secure communications are thereafter conducted (not shown). Such an exchange of an encryption key may allow access to the full range of programming options.

In contrast to the tap sensor and other sensors utilized in the prior art with implantable devices, in the present invention the tapping is not simply utilized to only wake the device.

Rather, the tapping is utilized to validate that communications about to be received by the implantable device 110. In other words, if tapping is normally utilized to wake the device additional, the additional tapping taught herein may be utilized to prepare the implantable device 110 for communication.

As depicted in FIG. 4, in other embodiments the implantable device 110 may determine whether a preset amount of time has expired (650) before declining the request for secure distance telemetry communication (630). For example, clock 170 may time stamp the receipt of the initial request for distance telemetry communication (600) and processor 150 may determine whether too great a time has passed before receiving the telemetry signal. If too great a time has passed then the request for communication is denied.

In still further embodiments, the implantable medical device 110 may include a type of proximity sensor to determine if the external device 10 is within a certain range before the stimulus is validated. Such an embodiment may represent a further check on the validity of the back door access initiated by the external stimulus. Still further embodiments may transmit an encryption key in response to a valid external stimulus such that secure communications can thereafter be conducted.

One of skill in the art will understand that components or steps described herein regarding a given embodiment or set of embodiments may readily be omitted, substituted, or added from, with, or to components or steps of other embodiments or sets of embodiments, as appropriate or desirable. It will be further understood that a computer readable medium containing instructions that when implemented effectuate an implantable medical device or external device to perform the methods described herein are contemplated.

One skilled in the art will appreciate that various aspects of the present disclosure can be practiced with embodiments other than those disclosed. The disclosed embodiments are presented for purposes of illustration and not limitation, and the present invention is limited only by the claims that follow.

What is claimed is:

1. A method associated with establishing communication between an implantable medical device and an external device, the method being performed by the implantable medical device and comprising:
receiving a short range external stimulus;
recognizing the external stimulus as a valid indication of impending authorized communication with an external device;
preparing to receive the authorized communication from the external device; and
conducting communication with the external device.

2. The method of claim 1, wherein preparing to receive authorized communication includes establishing a non-secure link with the external device.

3. The method of claim 1, wherein receiving short range external stimulus comprises receiving one or more taps in a pre-selected pattern.

4. The method of claim 3, wherein receiving short range external stimulus further comprises receiving one or more taps using an accelerometer.

5. The method of claim 1 wherein receiving the short range external stimulus is one or more of receiving a magnetic field, an audible signal, or an ultrasonic signal.

6. The method of claim 1, wherein preparing to receive authorized communication includes establishing a secure link with the external device.

7. The method of claim 6, wherein preparing to receive the authorized communication further comprises sending an encryption key to the external device.

8. The method of claim 4 further comprising communicating with the external device using the encryption key.

9. An implantable medical device comprising:
a tap sensor; and
a processor operably coupled to the tap sensor wherein the processor is configured to receive an external stimulus from the tap sensor and to recognize the external stimulus as a valid indicator of impending authorized communication.

10. The implantable medical device of claim 9, wherein the processor is further configured to establish non-secure communication via a radio frequency transceiver module if the external stimulus is valid.

11. The implantable medical device of claim 9, wherein the processor is further configured to establish secure communication via a radio frequency transceiver module if the external stimulus is valid.

12. The implantable medical device of claim 9, wherein the processor is further configured to send an encryption key through the radio frequency transceiver if the external stimulus is valid.

13. The implantable medical device of claim 9 wherein the tap sensor is an accelerometer.

14. An implantable medical device comprising:
a stimulus sensor; and
a processor operably coupled to the stimulus sensor wherein the processor is configured to receive an external stimulus from the stimulus sensor and to recognize the external stimulus as a valid indicator of impending authorized communication.

15. The medical device of claim 14 wherein the stimulus sensor is a magnetic field sensor.

16. The medical device of claim 14 wherein the stimulus sensor is a sound sensor that senses sound in the audible range.

17. The medical device of claim 14 wherein the stimulus sensor is a sound sensor that senses sound in the ultrasonic range.
